(19) United States
(12) Patent Application Publication
(10) Pub. No.: US 2010/0057167 A1
(43) Pub. Date: Mar. 4, 2010

(54) System and Method for the Interrogation of Implantable Medical Devices

(76) Inventors: Xander Evers, Amhem (NL); Jeroen Zuiderhoek, Duiven (NL)

Correspondence Address: MEDTRONIC, INC., 710 MEDTRONIC PARKWAY NE, MINNEAPOLIS, MN, 55432-9924, US

(21) Appl. No.: 12/203,042

(22) Filed: Sep. 2, 2008

(51) Int. Cl. A61N 1/36; A61B 5/07

(52) U.S. Cl. 607/60; 600/302

(57) ABSTRACT

A system and method are provided for interrogating an implantable medical device (IMD) using a platform-independent interrogation process with an interrogator device, such that the interrogation is initiated by the interrogator device without requiring information relating to the location of where the diagnostic data is stored in the memory of the IMD and without requiring information related to the type or format of the diagnostic data. By utilizing a platform-independent interrogation process, a generic universal interrogation process can be utilized that can be extended across IMDs and interrogator devices capable of having different platforms. Further, the platform-independent interrogation process allows an interrogation device to interrogate different types of IMDs, and also allows IMDs to be interrogated by different types of interrogator devices, thereby allowing IMDs and interrogator devices to be interchangeably used and easily updated without requiring entire interrogation system reconfigurations.

### TECHNICAL FIELD

The present invention relates to implantable medical devices (IMDs) and, more particularly, to a system and method for interrogating an IMD.

### BACKGROUND

Various types of devices have been developed for implantation into the human body to provide various types of health-related therapies and/or monitoring. Examples of such devices, generally known as implantable medical devices (IMDs), include cardiac pacemakers, cardioverter/defibrillators, cardiomyostimulators, cardiac event monitors, various physiological stimulators including nerve, muscle, and deep brain stimulators, various types of physiological monitors, and drug delivery systems, just to name a few. Some IMDs include varying amounts of electronic memory that may be used to store not only device operating and control software, but to store various types of patient and device-related data. In addition, some of these same IMDs may include signal processing and telemetry circuitry, which allows some or all of the data stored in the memory to be transmitted to a remote computer network or other communication node, and/or the device to receive and store data transmitted to it remotely from a computer network or other communication node.

In many cases, after an IMD has been implanted in a patient, the patient may need to have periodic follow-up visits with a doctor or other type of practitioner. Alternatively, or in addition to periodic follow-up visits, patients with IMDs may need to periodically initiate a communication with a doctor or other type of practitioner at a medical facility or clinic, or periodically initiate a remote communication, such as described above, between the IMD and a remote network or other communications node. These periodic visits and/or communications, allow doctors or other practitioners to check the IMD and patient to determine, for example, whether or not the IMD is operating as programmed or perhaps should be programmed differently. These periodic visits and/or communications also allow, among other things, doctors or other practitioners to analyze some or all of the data stored in and/or transmitted from the IMD. These data can provide the doctor or other practitioner with various types of physiological data about the patient, and may also be used to determine whether or not the IMD is functioning properly and whether the patient is responding as expected to the treatment applied by the IMD.

In many instances, the above-described remote monitoring occurs over the telephone infrastructure. In particular, a patient monitor device, which is located in the patient's home, may be connected to a telephone jack in the patient's home, and may include a monitor wand. The monitor wand may include a radio frequency (RF) antenna to receive the above-mentioned patient- and device-related data that may be transmitted by the IMD. Periodically, a remote station operator may call the patient monitor and request certain data to be transferred from the IMD to the remote station operator. The patient monitor will then interrogate the IMD to obtain certain data stored in the memory of the IMD, where the data obtained by the patient monitor is in turn transmitted to the remote station operator. The collection and transmission of such data allows a remote operator to view the results of the IMD interrogation to allow for remote monitoring and diagnosis.

In order to perform this interrogation of the IMD, conventional patient monitoring systems have required the patient monitor to record information relating to the contents of the IMD memory so that the patient monitor knows the portions of the IMD memory that need to be accessed in order to obtain the requested data. This knowledge and understanding of the contents of the IMD memory by the patient monitor has also conventionally required each patient monitor to be specially developed to interact with a respective IMDs.

### SUMMARY

In one or more embodiments, a system and method are provided for interrogating an implantable medical device (IMD) using a platform-independent interrogation process with an interrogator device, such that programs and modules operating on both the IMD and the interrogator device can be independent of each other. By utilizing a platform-independent interrogation process, a generic universal interrogation process can be utilized that can be extended across IMDs and interrogator devices capable of having different platforms. Further, the platform-independent interrogation process allows an interrogation device to interrogate different types of IMDs, and also allows IMDs to be interrogated by different types of interrogator devices, thereby allowing IMDs and interrogator devices to be interchangeably used and easily updated without requiring entire interrogation system reconfigurations.

In one or more embodiments, the method for interrogating an IMD using a platform-independent interrogation process includes interrogating the implantable medical device from the platform-independent interrogator device in order to transfer diagnostic data stored in a memory of the IMD to the interrogator device, such that the interrogation is initiated by the interrogator device without requiring information relating to the location of where the diagnostic data is stored in the memory of the IMD and without requiring information related to the type or format of the stored diagnostic data.

In one or more embodiments, the interrogation process is initiated by the interrogator device sending an interrogation request to the implantable medical device to initiate the interrogation. In response, historical interrogation information is retrieved from memory in the IMD and sent to the interrogator device. From the historical interrogation information, the interrogator device determines a temporal starting point and temporal range for certain desired diagnostic data to be retrieved from the IMD, where the temporal starting point and temporal range are sent to the IMD. The IMD then determines the locations in the implantable medical device memory in which the desired diagnostic data relating to the temporal starting point and temporal range are stored and forwards such memory locations to the interrogator device. The interrogator device determines a desired manner for reading the desired diagnostic data from such memory locations and communicates instructions to the IMD to read the desired diagnostic data from such memory locations in the determined desired manner. The IMD then reads the desired diagnostic data from its memory in the instructed manner and communicates the diagnostic data to the interrogator device.

In one or more embodiments, the interrogator device requests the IMD to capture a snapshot of a status of at least portions of the memory of the IMD either before or after or preferably before and after the transfer of diagnostic data from the memory of the IMD to the interrogator device. Once the interrogation has been completed, the interrogator device transmits a notification to the implantable medical device along with information relating to the completed interrogation to be stored with historical interrogation information stored on the implantable medical device. In one or more embodiments, the diagnostic data retrieved during the interrogation process and the memory snapshots are then communicated from the interrogator device to a diagnostic site or another component for further review and analysis.

### DRAWINGS

The above-mentioned features and objects of the present disclosure will become more apparent with reference to the following description taken in conjunction with the accompanying drawings wherein like reference numerals denote like elements and in which:

FIG. 1 illustrates components of the system for interrogating an implantable medical device in accordance with one or more embodiment of the present disclosure.

FIG. 2 is a block diagram illustration of exemplary configurations for the system for interrogating an implantable medical device in accordance with one or more embodiment of the present disclosure.

FIG. 3 is an operational flow diagram illustrating a method for interrogating an implantable medical device in accordance with one or more embodiment of the present disclosure.

FIG. 4 is a operational sequence diagram illustrating a method for interrogating an implantable medical device in accordance with one or more embodiment of the present disclosure.

FIG. 5 is a block diagram illustrating the various components of the implantable medical device and interrogator device configured to operate in accordance with one or more embodiments of the present disclosure.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

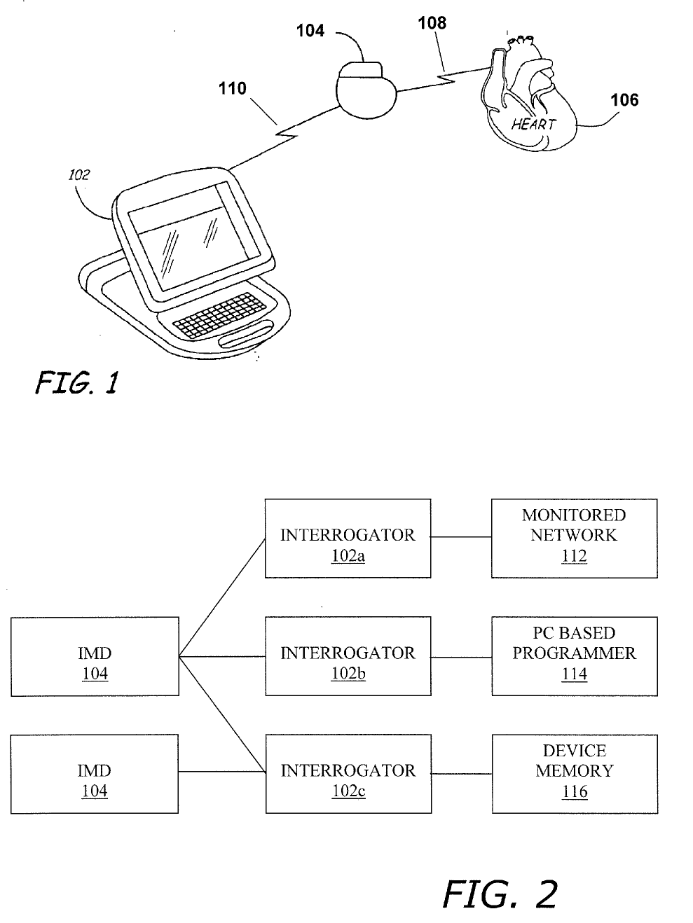

A system and method are provided for interrogating an implantable medical device (IMD) using a platform-independent interrogation process with an interrogator device. FIG. 1 illustrates a simplified schematic view of one type of implantable medical device (“IMD”) 104 that can be implanted within a human body and interrogated by a platform-independent interrogator device 102 in accordance with one or more embodiments. In one embodiment, IMD 104 may comprise a hermetically sealed enclosure and connector module for coupling IMD 104 to electrical leads and other physiological sensors arranged within a human body, such as pacing and sensing leads 108 connected to portions of a heart 106 for delivery of pacing pulses to a patient's heart 106 and sensing the performance of the heart 106. While IMD 104 is depicted in a pacemaker device configuration in FIG. 1, it is understood that IMD 104 may comprise any type of implanted, subcutaneous or external medical device. IMD 104 collects and stores diagnostic data from one or more sensors for sensing physiological signals, conditions or parameters occurring in the patient in which IMD 104 is implanted.

In one or more embodiments, platform-independent interrogator device 102 is configured to communicate with IMD 104 through a communication pathway 110 for interrogating the memory of IMD 104 in order to retrieve or download the stored diagnostic data for medical and/or device diagnosis. In one or more embodiments, the interrogator device 102 may be a dedicated device, a specially configured personal computer, or a software module executed on a dedicated instrument or specially configured personal computer. In one or more embodiments, interrogator device 102 may be included within a portable device wearable or capable of being carried by the patient. In one or more embodiments, interrogator device 102 may comprise a monitoring device in a physician's office or the patient's home, such as the Medtronic CareLink® Network monitor, that collects information from IMDs implanted in patients and communicates such information to remote clinicians through the Internet, phone lines or wireless networks. Carelink is a registered trademark of Medtronic, Inc. of Minneapolis, Minn.

Home monitoring networks, such as the Medtronic CareLink Network, enable patients to transmit data from their IMD 104, as instructed by their physician, using a portable monitor (i.e., interrogator device 102) that is connected to the Internet, phone lines or wireless networks. Within a short period of time after an interrogation, the patient's physician and other caregivers can review and analyze the diagnostic data retrieved from IMD 104. This use of the interrogation process provides an efficient, safe and convenient way for a multitude of caregivers to optimize patient care by remotely monitoring the condition of their patients and, if needed, make adjustments to device operation, medication or prescribe additional therapy.

In one or more embodiments, the system and method for interrogating an IMD 104 use a platform-independent interrogation process with interrogator device 102, such that programs and modules operating on both IMD 104 and interrogator device 102 can be independent of each other. For example, interrogation software running on interrogator device 102 and device software running on IMD 104 can be designed to be independent, thereby allowing a generic universal interrogation process to be implemented to extend across IMDs and interrogator devices capable of having different platforms. As used herein, the term “platform” shall refer to types of devices, products and/or software used for IMD 104 or interrogator device 102, and may include versions of the devices, products and/or software. As such, the platform-independent interrogation process of the present system and method allows an interrogation device 102 to interrogate different types of IMDs 104, and also allows IMDs 104 to be interrogated by different types of interrogator devices 102, thereby allowing IMDs 104 and interrogator devices 102 to be interchangeably used and easily updated without requiring entire interrogation system reconfigurations.

By way of example, FIG. 2 illustrates a block diagram of exemplary configurations in which different interrogator devices 102a, 102b, 102c may be used to interrogate the same IMD 104. For example, interrogator device 102a may comprise an in-home monitor that is connected to communicate diagnostic data to a monitored network 112 (e.g., the Medtronic CareLink® Network), interrogator device 102b may comprise a physician monitor that is connected to communicate diagnostic data to a PC-based programmer 114 or other device programmer for configuring IMD 104, and/or interrogator device 102c may comprise a dedicated interrogation device having a memory 116 for storing retrieved diagnostic data for later analysis by a caregiver or for device or medical diagnosis. Each of the interrogator devices 102a, 102b, 102c may comprise the same type of interrogator device or may be different types of interrogator device, but each will be able to interrogate IMD 104 based on the platform-independent interrogation process described herein. Furthermore, each interrogator device 102 may be able to interrogate a plurality of different IMDs 104. By utilizing a platform-independent interrogation process, a generic universal interrogation process can be utilized that can be extended across IMDs 104 and interrogator devices 102 capable of having the same or different platforms, thereby allowing IMDs 104 and/or interrogator devices 102 to be interchangeably used and easily updated without requiring entire interrogation system reconfigurations. This can be especially beneficial in the case of an IMD 104 that is implanted within a patient and which has limited accessibility, where updates or changes to interrogator devices 102 can be made without necessarily requiring the IMD 104 to be similarly updated or changed.

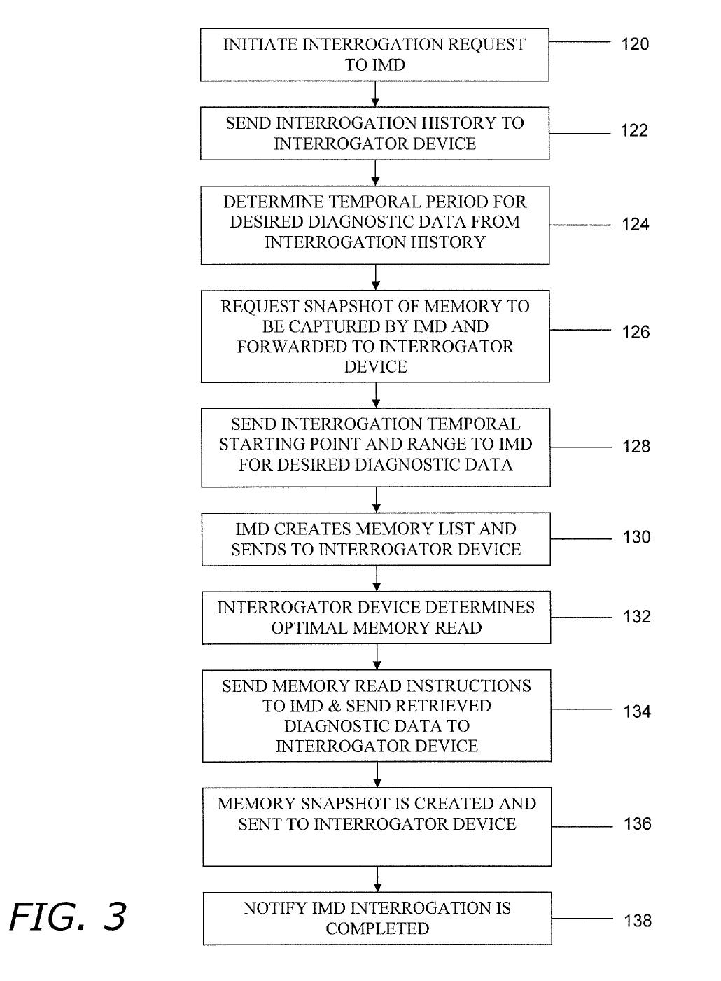

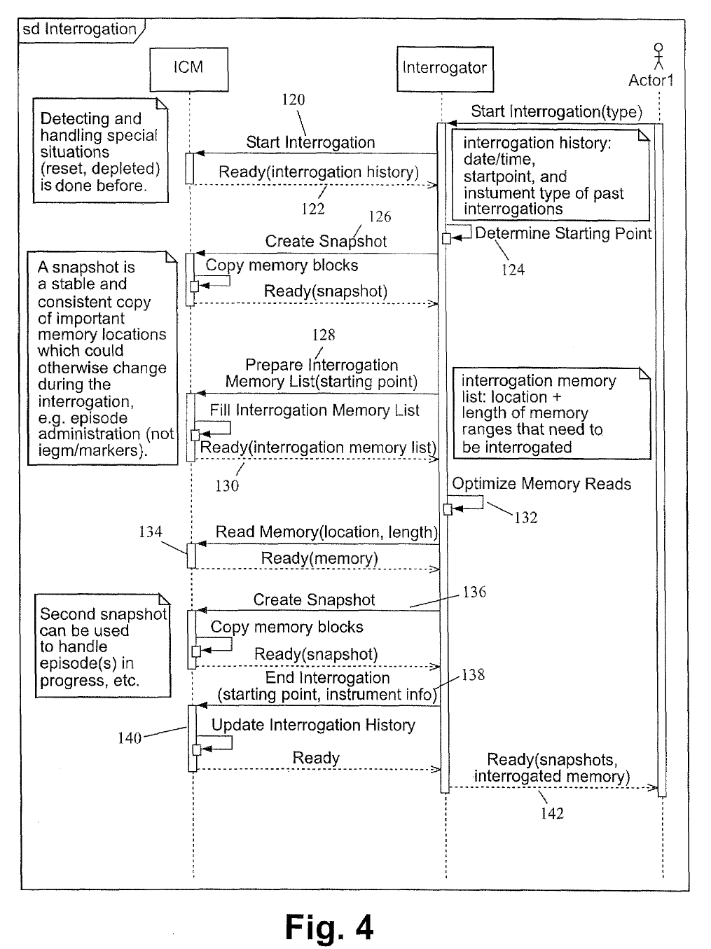

Referring now to FIG. 3, an operational flow diagram is illustrated in accordance with one or more embodiments for a method for interrogating an IMD 104 using a platform-independent interrogation process with an interrogator device 102. The operational flow diagram of FIG. 3 will be described with further reference to an operation sequence diagram showing the operations and exchange of information during the platform-independent interrogation process as illustrated in FIG. 4.

Initially, interrogator device 102 will be triggered to initiate the interrogation process, where the trigger can be derived from a variety of possible sources, including but not limited to a user or caregiver initiating the request, a scheduled or timed trigger event, or based in response to certain data interpreted by interrogator device 102, etc. Interrogator device 102 sends an interrogation request to IMD 104 in operation 120 to request or notify IMD 104 to begin the interrogation. At this point in time, interrogator device 102 has no knowledge or information relating to the locations of where the diagnostic data is stored in the memory of IMD 104. Further, there is no requirement for interrogator device 102 to have information relating to the type, kind or format of the diagnostic data stored in the memory of IMD 104. Thus, in response to the interrogation request, IMD 104 retrieves historical interrogation information stored in its memory and sends the historical interrogation information to interrogator device 102 in operation 122. The historical interrogation information may include information relating to prior completed interrogations, such as but not limited to the date and time of prior interrogations, temporal start points, stop points and periods for which diagnostic data was transferred for prior interrogations, and an identifier of the type of instrument, device or platform used in prior interrogations. In one or more embodiments, IMD 104 is entirely responsible for maintaining its own historical interrogation information, thereby adding to the platform independence of the interrogator device 102 since interrogator device 102 is not required to have any prior knowledge of the memory contents of IMD 104 (including information relating to prior diagnostic data that has been transferred during prior interrogations).

From the historical interrogation information that is received, interrogator device 102 determines in operation 124 for which temporal time period diagnostic data needs to be interrogated (e.g., from a temporal starting point until present). In one or more embodiments, the temporal time period includes a temporal starting point and temporal range for certain desired diagnostic data to be retrieved from the memory of IMD 104. Interrogator device 102 may take other information into account when determining the temporal time period (e.g., transmission failures in case of a home monitor).

In one or more embodiments, interrogator device 102 requests IMD 104 to capture a snapshot of a status of at least portions of the memory of IMD 104 in operation 126. A snapshot is a stable and consistent copy of important memory locations which could otherwise change and become inconsistent during the interrogation process (e.g., episode administration). For instance, the administration (memory pointers) of available episodes of diagnostic data is copied, but not the diagnostic data for the episodes itself. As such, the snapshot is a copy of those memory locations containing administrative information about the status of certain diagnostics, as opposed to a copy of the actual diagnostic data. One specific problem that is addressed is that during an interrogation, which can take a significant amount of time (e.g., several minutes), new episodes may begin or/and an episode in progress may end in which new diagnostic data is recorded in the memory of IMD 104. Memory snapshots can be taken both before the interrogation process begins and after the interrogation process ends, where the memory snapshots can be compared against each other and analyzed to determine if the status of the memory contents have changed during the interrogation process. Thus, IMD 104 captures a memory snapshot while knowing which important memory locations should be included in the memory snapshot, and IMD 104 sends the memory snapshot to interrogator device 102 in operation 126 for interrogator device 102.

The temporal time period information including the temporal starting point and temporal range are sent to IMD 104 in operation 128. Since interrogator device 102 has no knowledge regarding the specific contents of the memory of IMD 104, it is IMD 104 that then determines the relevant locations and/or ranges in the memory of IMD 104 in which the desired diagnostic data relating to the temporal starting point and temporal range are stored. IMD 104 then forwards an interrogation memory list to interrogator device 102 in operation 130, where the interrogation memory list includes the location and length of memory ranges that need to be interrogated to have all of the diagnostic data retrieved for the desired temporal time period.

From the received interrogation memory list, interrogator device 102 determines a desired or optimal manner for reading the desired diagnostic data from the memory locations of IMD 104 in operation 132. This desired or optimal manner may depend on a variety of possible factors, including but not limited to the memory layout and the telemetry protocol and capabilities, and may result in reading some extra memory locations that are not necessarily needed for the desired diagnostic data. The optimized memory read instructions are sent to IMD 104 in operation 134, where IMD 104 then follows such instructions in reading and retrieving the desired diagnostic data from memory locations in the manner instructed by interrogator device 102. The retrieved diagnostic data is further communicated to interrogator device 102 in operation 134.

In one or more embodiments, interrogator device 102 requests IMD 104 to capture another memory snapshot in operation 136 that is substantially identical to the memory snapshot captured in operation 126. This second memory snapshot provides administrative status information relating to important memory locations in IMD 104 after the diagnostic data has been transferred to interrogator device 102. The second memory snapshot is further sent to interrogator device 102 in operation 136. The first and second memory snapshots captured in operations 126 and 136 can then subsequently be used when analyzing the diagnostic data in a comparison operation that compares the first memory snapshot against the second memory snapshot to determine if the status of the memory contents have changed during the interrogation process. It should be noted that this comparison of the memory snapshots is performed after the interrogation process has been completed when analyzing the diagnostic data to assist in determining whether the retrieved diagnostic data is valid. If the comparison of the two memory snapshots indicates that the certain portions of the diagnostic memory may have been (at least partially) overwritten with new data during the memory reads, then it may be determined that those portions of the retrieved diagnostic data that have been communicated to interrogator device 102 are invalid or unreliable. To the contrary, all of the retrieved diagnostic data can be deemed valid and reliable if the comparison of the two memory snapshots indicates that the status of the memory contents did not change during the interrogation process.

In one or more embodiments, once it is determined by interrogator device 102 that the interrogation is complete and all required diagnostic data and memory snapshots have been received from IMD 104, interrogator device 102 sends IMD 104 a notification message that the interrogation is finished in operation 138, and further provides information relating to the completed interrogation for IMD 104 to store with historical interrogation information stored on the implantable medical device. For example, this information relating to the completed interrogation could include an indication of the device or instrument type of interrogator device 102 and temporal time period information relating to the interrogation. As long as the interrogation procedure was not stopped or interrupted prior to receiving such notification from interrogator device 102, IMD 104 will then update the historical interrogation information stored in its memory with the information from the recently completed interrogation in operation 140. In one or more embodiments, the memory snapshots and interrogation memory list that are created by IMD 104 are temporary data structures that are not otherwise used by IMD 104, where such temporary data structures may be deleted or discarded after the interrogation is complete or if the interrogation was interrupted prior to completion. Further, the historical interrogation information is not updated by IMD 104 if the interrogation was interrupted prior to completion.

In one or more embodiments, the diagnostic data retrieved during the interrogation process and the memory snapshots and other possible relevant information (e.g., temporal time period information relating to the interrogation) may then be communicated from interrogator device 102 to a diagnostic site or another component for further review and analysis in operation 142. In one or more embodiments, this transmission of diagnostic data and other information may also occur at various stages, such as when the interrogation is complete but prior to notifying IMD 104 of its completion or in stages as information is received by interrogator device 102.

Using a platform-independent interrogation process as described herein, interrogator device 102 is not required to have any knowledge or information regarding the memory layout of IMD 104 or about the diagnostic data that is transferred, as such diagnostic information can simply be treated as memory contents to interrogator device 102. This allows interrogator device 102 to be platform-independent, such that interrogator device 102 does not need to be changed or modified when new or updated IMDs 104 are to be interrogated. Similarly, the platform-independence extends to IMD 104 so that IMDs 104 do not need to be changed or modified when new or updated interrogator devices 102 are used for interrogation. A single IMD 104 can also be interrogated at different times by different types of interrogator devices 102 (e.g., the patient's home monitor and then separately by a programmer in a physician's office).

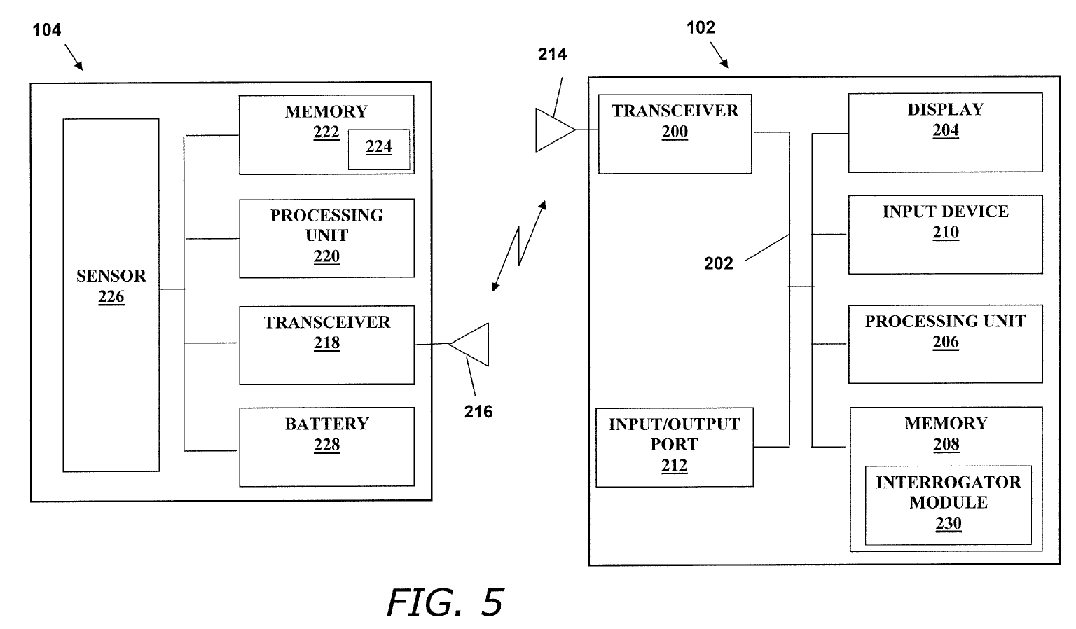

Referring now to FIG. 5, a block diagram illustration is provided showing the constituent components of IMD 104 and interrogator device 102 in accordance with one or more embodiments having a microprocessor-based architecture. IMD 104 is shown as including a transceiver 218, at least one sensor 226 for sensing physiological signals, processor or controller 220, memory 222, interrogation module 224 stored in memory 222, battery 228 and other components as appropriate to produce the desired functionalities of the device.

Processor 220 may be implemented with any type of microprocessor, digital signal processor, application specific integrated circuit (ASIC), field programmable gate array (FPGA) or other integrated or discrete logic circuitry programmed or otherwise configured to provide functionality as described herein. Processor 220 executes instructions stored in memory 222 to provide functionality as described herein, such as those instructions associated with an interrogation module 224 stored in memory 222. Instructions provided to processor 220 may be executed in any manner, using any data structures, architecture, programming language and/or other techniques. Memory 222 is any storage medium capable of maintaining digital data and instructions provided to processor 220 such as a static or dynamic random access memory (RAM), read-only memory (ROM), non-volatile random access memory (NVRAM), electrically erasable programmable read-only memory (EEPROM), flash memory, or any other electronic, magnetic, optical or other storage medium.

In operation, IMD 104 obtains data from physiological signals via electrodes and/or sensors 226 deployed on leads 108 and/or other sources. This data is provided to processor 220, which suitably analyzes the data, stores appropriate data in memory 222 and/or provides a response or report as appropriate. Communication between IMD 104 and another device can occur via telemetry, such as a long-distance telemetry system through the transceiver 218 or a telemetry module. Transceiver 218 may comprise any unit capable of facilitating wireless data transfer between IMD 104 and interrogator device 102 and/or another transceiver device. Transceiver 218 and a transceiver 200 in interrogator device 102 are respectively communicatively coupled through antennas 216 and 214 for facilitating the wireless data transfer. Transceiver 218 may be configured to perform any type of wireless communication. For example, transceiver 218 may send and receive radio frequency (RF) signals, infrared (IR) frequency signals, or other electromagnetic signals. In the case of electromagnetic signals, antennas 214 and 216 may comprise coils for transmitting and receiving signals when positioned adjacent to one another. Any of a variety of modulation techniques may be used to modulate data on a respective electromagnetic carrier wave. Alternatively, transceiver 218 may use sound waves for communicating data, or may use the patient's tissue as the transmission medium for communicating with a programmer positioned on the patients skin. In any event, transceiver 218 facilitates wireless data transfer between IMD 104 and interrogator device 102. Other types of wired communications may also occur when IMD 104 is alternatively configured as an external medical device or contains wired communication channels that extend from within the patient to points outside of the patient.

IMD 104 includes at least one sensor 226 configured to sense at least one physiological signal or condition, from which a physiological parameter can be determined and from which diagnostic data is obtained. Sensor(s) 226 can monitor electrical, mechanical, chemical, or optical information that contains physiological data of the patient and can utilize any source of physiological signals used for physiological events or conditions. For example, sensor(s) 226 may comprise a heart sensor, such as those in the MDT Reveal® system, commercially available from Medtronic of Minneapolis, that is capable of sensing cardiac activity, electrocardiograms, heart rate, or the like. Reveal is a registered trademark of Medtronic, Inc. of Minneapolis, Minn. The diagnostic data obtained from sensor(s) 226 is stored in memory 222 to be retrieved during interrogation.

With further reference to FIG. 5, interrogator device 102 includes an antenna 214, coil or wired input for communicating data and other signals between interrogator device 102 and IMD 104. Data is received from IMD 104 through antenna 214, which is connected to transceiver 200 that serves to receive and transmit communication signals through antenna 214. The demodulated signals are applied in parallel or serial digital format to input/output (I/O) unit or bus 202, where they in turn may be applied to a display or screen 204, provided to processor 206 and/or memory 208. In some embodiments, display 204 may include other types of interface devices capable of communicating information to the patient (e.g., a speaker device or other output device). Processor 206 includes any type of microprocessor, digital signal processor, application specific integrated circuit (ASIC), field programmable gate array (FPGA) or other integrated or discrete logic circuitry programmed or otherwise configured to control operating of interrogator device 102 and provide functionality as described herein. In one or more embodiments, processor 206 executes instructions stored in memory 208 to provide functionality as described herein. In one or more embodiments, instructions may be stored in memory 208 for operating an interrogation module 230 or program that allows the platform-independent interrogation procedures to be performed.

In one or more embodiments, interrogator device 102 includes an input device 210 that allows data, commands or selections to be input into interrogator device 102 by a patient, physician or clinician. Input device 210 may include, but is not limited to, at least one of the following: a keyboard, keypad, track ball, mouse, touch-sensitive displays, push buttons, magnetic readers, RF readers, tablets, styluses, microphones, voice recognizers, handwriting recognizers and any other device that allows a patient, physician or clinician to input data to external device. Processor 206 controls operation of display 204 and is responsive to commands received from input device 210. Memory 208 is suitable for storing interrogation module 230, diagnostic data and snapshots received from IMD 104 during the interrogation along with other information received by interrogator device 102. Interrogator device 102 may further include an input/output port 212 for connecting , interrogator device 102 to other devices, communication networks (e.g., Carelink), phone lines, wireless devices, etc.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method of interrogating an implantable medical device, comprising:
providing a platform-independent interrogator device; and
interrogating an implantable medical device from the platform-independent interrogator device to transfer diagnostic data stored in a memory of the implantable medical device to the interrogator device, such that the interrogation is initiated by the interrogator device without requiring information relating to the memory location or type of diagnostic data stored in the memory of the implantable medical device.

2. The method of claim 1, wherein the interrogation of the implantable medical device includes communicating an interrogation request from the interrogator device to the implantable medical device to initiate the interrogation and receiving in response to the interrogation request historical interrogation information transmitted from the implantable medical device.

3. The method of claim 2, further comprising:
determining at the interrogator device a temporal starting point and temporal range for certain desired diagnostic data to be transferred during the interrogation based on an analysis of the historical interrogation information;
transmitting the temporal starting point and temporal range to the implantable medical device; and
receiving from the implantable medical device information relating to locations in the implantable medical device memory in which the desired diagnostic data relating to the temporal starting point and temporal range is stored.

4. The method of claim 3, further comprising:
determining at the interrogator device a desired manner for reading the memory locations associated with the desired diagnostic data;
communicating instructions to the implantable medical device to read the desired diagnostic data from their memory locations in the determined desired manner; and
receiving at the interrogator device the desired diagnostic data that has been read from the memory locations of the implantable medical device.

5. The method of claim 1, further comprising:
Transmitting a notification from the interrogator device to the implantable medical device when the interrogation has been completed; and
communicating information from the interrogator device to the implantable medical device relating to the completed interrogation to be stored with historical interrogation information on the implantable medical device.

6. The method of claim 2, further comprising:
requesting and receiving at the interrogator device a snapshot of a status of at least portions of the memory of the implantable medical device before and after the transfer of diagnostic data stored in the memory of the implantable medical device to the interrogator device.

7. The method of claim 6, further comprising communicating the diagnostic data and the snapshots received by the interrogator device to a diagnostic site for further analysis.

8. A method of operating an implantable medical device in response to an interrogation by an interrogator device, comprising:
providing an implantable medical device that is platform-independent from an interrogator device;
recording diagnostic data in a memory of the implantable medical device;
operating the implantable medical device to transfer diagnostic data stored in a memory of the implantable medical device to at least one platform-independent interrogator device in response to an interrogation by the interrogator device; and
storing historical interrogation information relating to at least one completed interrogation in the memory of the implantable medical device.

9. The method of claim 8, further comprising:
receiving an interrogation request from the interrogator device sent to the implantable medical device to initiate an interrogation;
retrieving the historical interrogation information from memory; and
transmitting the retrieved historical interrogation information to the interrogator device.

10. The method of claim 8, further comprising:
receiving a request from the interrogator device for a snapshot of a status of at least portions of the memory of the implantable medical device before and after the transfer of diagnostic data stored in the memory of the implantable medical device to the interrogator device;
generating corresponding snapshots and sending the snapshots to the interrogator device.

11. The method of claim 8, further comprising:
receiving from the interrogator device a temporal starting point and temporal range for certain desired diagnostic data to be transferred during the interrogation based on an analysis of the historical interrogation information;
determining respective locations in the memory in which the desired diagnostic data relating to the temporal starting point and temporal range are stored; and
transmitting the determined memory locations for the desired diagnostic data to the interrogator device.

12. The method of claim 11, further comprising:
receiving instructions from the interrogator device relating to a desired manner for the implantable medical device to read the memory locations associated with the desired diagnostic data;
retrieving the desired diagnostic data from memory according to the desired read operations; and
transmitting the retrieved desired diagnostic data to the interrogator device.

13. The method of claim 12, further comprising:
receiving a notification from the interrogator device that the interrogation has been completed, where such notification includes information relating to the completed interrogation;
storing the information relating the completed interrogation with the historical interrogation information stored on the implantable medical device.

14. A platform-independent interrogator device for interrogating an implantable medical device, comprising:
a memory;
an interrogation module stored in memory; and
a processor for executing the interrogation module for interrogating an implantable medical device from the platform-independent interrogator device to transfer diagnostic data stored in a memory of the implantable medical device to the interrogator device, such that the interrogation is initiated by the interrogator device without requiring information relating to the memory location or type of diagnostic data stored in the memory of the implantable medical device.

15. The device of claim 14, wherein the processor causes an interrogation request to be communicated from the interrogator device to the implantable medical device to initiate the interrogation and wherein the device receives in response to the interrogation request historical interrogation information transmitted from the implantable medical device.

16. The device of claim 15, wherein the processor further executes the interrogation module for:
determining at the interrogator device a temporal starting point and temporal range for certain desired diagnostic data to be transferred during the interrogation based on an analysis of the historical interrogation information;
transmitting the temporal starting point and temporal range to the implantable medical device; and
receiving from the implantable medical device information relating to locations in the implantable medical device memory in which the desired diagnostic data relating to the temporal starting point and temporal range is stored.

17. The device of claim 16, wherein the processor further executes the interrogation module for:
determining at the interrogator device a desired manner for reading the memory locations associated with the desired diagnostic data;
communicating instructions to the implantable medical device to read the desired diagnostic data from their memory locations in the determined desired manner; and
receiving at the interrogator device the desired diagnostic data that have been read from the memory locations of the implantable medical device.

18. The device of claim 14, wherein the processor further executes the interrogation module for:
issuing a notification to the implantable medical device when the interrogation has been completed; and
communicating information to the implantable medical device relating to the completed interrogation to be stored in an interrogation history on the implantable medical device.

19. The device of claim 14, wherein the processor further executes the interrogation module for requesting and receiving at the interrogator device a snapshot of a status of at least portions of the memory of the implantable medical device before and after the transfer of diagnostic data stored in the memory of the implantable medical device to the interrogator device.

20. The device of claim 19, wherein the processor further executes the interrogation module for communicating the diagnostic data and the snapshots received by the interrogator device to a diagnostic site for further analysis.

21. An implantable medical device, comprising:
a memory;
an interrogation module stored in memory; and
a processor for executing the interrogation module for:
providing an implantable medical device that is platform-independent from an interrogator device;
recording diagnostic data in a memory of the implantable medical device;
operating the implantable medical device to transfer diagnostic data stored in a memory of the implantable medical device to at least one platform-independent interrogator device in response to an interrogation by the interrogator device; and
storing historical interrogation information relating to at least one completed interrogation in the memory of the implantable medical device.

22. The implantable medical device of claim 21, wherein the processor further executes the interrogation module for:
receiving an interrogation request from the interrogator device sent to the implantable medical device to initiate an interrogation;
retrieving the historical interrogation information from memory; and
transmitting the retrieved historical interrogation information to the interrogator device.

23. The implantable medical device of claim 21, wherein the processor further executes the interrogation module for:
receiving a request from the interrogator device for a snapshot of a status of at least portions of the memory of the implantable medical device before and after the transfer of diagnostic data stored in the memory of the implantable medical device to the interrogator device;
generating corresponding snapshots and sending the snapshots to the interrogator device.

24. The implantable medical device of claim 21, wherein the processor further executes the interrogation module for:
receiving from the interrogator device a temporal starting point and temporal range for certain desired diagnostic data to be transferred during the interrogation;
determining respective locations in the memory in which the desired diagnostic data relating to the temporal starting point and temporal range are stored; and
transmitting the determined memory locations for the desired diagnostic data to the interrogator device.

25. The implantable medical device of claim 24, wherein the processor further executes the interrogation module for:
receiving instructions from the interrogator device relating to a desired manner for the implantable medical device to read the memory locations associated with the desired diagnostic data;
retrieving the desired diagnostic data from memory according to the desired read operations; and
transmitting the retrieved desired diagnostic data to the interrogator device.

26. The implantable medical device of claim 25, wherein the processor further executes the interrogation module for:
receiving a notification from the interrogator device that the interrogation has been completed, where such notification includes information relating to the completed interrogation;
storing the information relating the completed interrogation with the historical interrogation information stored on the implantable medical device.
